(19) United States
(12) Patent Application Publication
(10) Pub. No.: US 2025/0110138 A1
(43) Pub. Date: Apr. 3, 2025

(54) TAGGED COMPOUNDS FOR DETECTION AND ASSAY OF SMALL MOLECULES

(71) Applicant: Aveta.Life Inc., Hoboken, NJ (US)

(72) Inventors: Natasha Shtraizent, Weehawken, NJ (US); Lina Freage, Weehawken, NJ (US); Henry Grage, Weehawken, NJ (US)

(21) Appl. No.: 18/906,231

(22) Filed: Oct. 4, 2024

Related U.S. Application Data
(63) Continuation of application No. 18/099,177, filed on Jan. 19, 2023.
Application No. 18/099,177 is a continuation of application No. 17/740,094, filed on May 9, 2022.

(51) Int. Cl. G01N 33/74; G01N 33/58

(52) U.S. Cl. CPC G01N 33/743; G01N 33/582; G01N 2470/10

(57) ABSTRACT

Probes that are versatile, easy to use, and provide rapid results for detecting and quantifying levels of small molecules that include steroids, hormones, antibodies, aptamers and enzymes such as various steroidal hormones like estrogen, progesterone and testosterone in samples. This is particularly useful in home and clinical settings. A probe useful in competitive assays includes a competitive ligand bound to a linker molecule bound to a detectable tag. The linker may be chemical, DNA or a combination of both.

### INCORPORATION BY REFERENCE OF SEQUENCE LISTING

This application contains a Sequence listing that has been submitted in a computer readable format and is hereby incorporated by reference in its entirety. The XML file, created on Oct. 3, 2024 is named 68272US06 and 18,645 bytes in size.

### RELATED APPLICATIONS

This application is a continuation of U.S. application Ser. No. 18/099,177 filed Jun. 14, 2024, which is a continuation of U.S. patent application Ser. No. 17/740,094, filed May 9, 2022 which are incorporated herein by reference in their entirety.

### BACKGROUND OF THE INVENTION

### Field of the Invention

The present invention relates generally to the field of hormonal assays and more particularly to tagged compounds that can include DNA probes for the detection and assay of human steroidal hormones and other small molecules.

### Description of the Problem Solved

Hormones are molecules produced by the body that function primarily as chemical messengers. They travel via the bloodstream from points of origin such as glands to tissues and organs throughout the body. A hormonal assay is a test that is performed on a blood or serum sample to measure the level of various specific hormones. Various hormones may be proteins or steroids. In particular, estrogens and progestins are common endogenous steroidal sex hormones.

Estrogens and progestins produce numerous physiological actions in both women and men. Female neuroendocrine actions generate estrogens and progestins involved in the control of ovulation and the cyclical preparation of the reproductive tract for fertilization and implantation. Estrogens and progestins are also commonly used for contraception and menopausal hormone replacement therapy.

A hormonal assay is a test that is performed on a blood sample to measure the level of various specific hormones. Several devices known in the art are approved for the detection of hormones, for example: lateral flow and Elisa. These devices rely on tagged compounds (probes) that compete for a target with endogenous levels of the hormones, for example in serum or plasma. More sensitive and accurate methods, such as Liquid Chromatography and Mass Spectrometry (LC-MS/MS), do not rely on competitive probes, but are not feasible for general clinical or consumer use. Prior art probes for consumer devices poorly estimate meaningful levels of hormones and/or are not sensitive. Further, these probes are not modular or easily multiplexed or amenable for signal amplification.

In general, Elisa is a plate-based assay that can detect soluble substances such as peptides, proteins, antibodies and hormones. For example, in one version of Elisa, a test for a particular antigen, which is a large target macro-molecule, includes a target specific antibody that is immobilized to a well in the plate. The antigen in the test sample specifically binds to the antibody. Then, tagged enzymes, which can be detected by various methods such as fluorescence, are caused to bind to the antigen-antibody complex allowing detection and quantification of the level of antigen in the sample.

A competitive assay is one where a competitive tagged molecule similar to the actual target molecule is mixed with a sample and compete for a substrate. The two similar molecules compete for an primary substrate (i.e. antibody, enzyme or other receptor) that attach to a secondary substrate immobilized on the plate. The tagged competitive molecule is added to an unknown sample in the coated well. Then the primary substrate is exposed to the mixture, incubated, washed, and visualized for comparison to a reference standard, or standard curve. When concentration of the target is low, more tagged competitive molecules bind; when concentration of the target is high, more of the target molecules bind. A high signal level indicates a low target concentration, whereas a low signal level indicates a high target concentration. With calibration, this type of test can be quantitative.

There is a need to in the art to identify compound probes that accurately detect and quantitatively estimate small molecules such as, but not limited to, steroids and steroidal hormones; in particular it would be extremely advantageous to quickly and accurately measure hormone levels. These compound probes should also provide a modular design for multiplexing in assays such as lateral flow or Elisa. These compounds probes would be useful to monitor levels that improve contraception and menopausal hormone therapy. The present invention addresses this need, namely providing a small molecule assay.

### SUMMARY OF THE INVENTION

The present invention relates to probes that are easy to use and provide rapid results for detecting and quantifying levels small molecules such as various steroidal hormones like estrogen, progesterone and testosterone in samples. This is particularly useful in home and clinical settings.

The present invention provides generally a compound of the formula (I), or a salt, solvate, isotopically labeled derivative, stereoisomer, tautomer, or geometric isomer thereof, and any mixtures thereof having the structure:

(competitive ligand)-LINKER-(signaling molecule (tag)). (I)

A competitive assay is shown in FIG. 1. The process takes place on a plate or other substrate 1. The plate is coated with a secondary receptor 10 such as an antibody. A receptor molecule 2 such as, but not limited to, a primary antibody sensitive to a particular small target molecule 3 such as a hormone is in solution near a plate, well or other substrate. The competitive ligand 7 is attached to a detectable tag 8 through a chemical and/or DNA linker 9. The competitive ligand binds 6 to a receptor 2 in solution so as to compete with the small target molecule 3. The bound complexes 11 then bind to the secondary molecule on the plate. The signaling molecule (tag) 8 allows detection, while the LINKER 9 is selected such that it allows for the compound to bind to a target and simultaneously facilitate detection. After flushing off unbound products, the detection signal is measured. A large signal results from a large number of bound probes which indicates a lower concentration of the target; a small signal results from a large number of bound target molecules which indicates a larger concentration of target molecules.

In an alternative embodiment shown in FIG. 2, the present invention provides a compound of the formula (II), or a salt, solvate, isotopically labeled derivative, stereoisomer, tautomer, or geometric isomer thereof, and any mixtures thereof that includes deoxyribonucleic acid (DNA):

(competitive ligand)-LINKER-(signaling molecule (tag)). (I)

where -(DNA) represents a single-strand sequence of DNA 21 bound to a first compound such as the competitive ligand 20, and (cDNA)-22 is the single strand complement of the DNA 21 which is bound to second compound such as a signaling molecule 24. The competitive ligand 20 can be attached to the cDNA or DNA by a suitable chemical linker 23. The two DNA segments are linked typically linked together by hydrogen bonds.

During the assay, the competitive ligand binds to a target such that it competes with a relevant small molecule such as a target hormone. The DNA itself either allows direct detection, or the DNA binds a complementary DNA tag that allows detection (cDNA-tag). An optional chemical LINKER may be bonded between the DNA and either the ligand or the signaling molecule. The DNA linker and chemical linker (if used) are selected such that they allow for the compound to bind to the target and simultaneously facilitate detection.

### DESCRIPTION OF THE FIGURES

Attention is now directed to several drawings that illustrate features of the present invention.

FIG. 1 shows a schematic diagram of a competitive assay using embodiments of probes of the present invention.

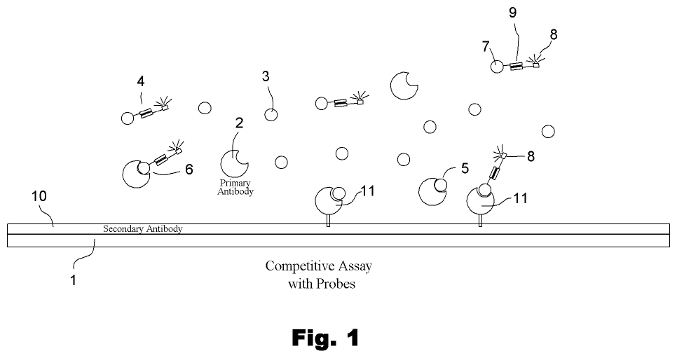

FIG. 2 shows an embodiment of a competitive probe with a DNA linker.

FIGS. 3A-3B show embodiments of competitive probes with either multiple competitive ligands or multiple signaling molecules.

FIG. 4 shows a competitive probe molecule that uses an 18 mer DNA sequence bound to its DNA complement that targets progesterone.

FIG. 5 shows a chemical linker bound to a competitive ligand.

FIG. 6A shows the chemical structure of progesterone.

FIG. 6B shows an embodiment of a competitive ligand for progesterone.

Several figures and illustrations have been provided to aid in understanding the present invention. The scope of the present invention is not limited to what is shown in the figures.

### DETAILED DESCRIPTION OF THE PREFERRED EMBODIMENTS

The present invention relates to bifunctional compounds that efficiently facilitate the in vitro detection of small molecules such as specific hormones. These can be, but are not limited to, estrogens and progestins. Assay methods can be any technique that relies on the in vitro detection such as, but not limited to, lateral flow or Elisa. The present invention detects the presence of and quantity of a target molecule in a sample.

### Definitions

A small molecule is a molecule that is non-peptidyl, i.e., it is not generally considered a peptide (e.g. if it contains amino acids, in general, it comprises fewer than 4 amino acids). It can be a steroid, enzyme, antibody or protein. A small molecule typically has a molecular weight that is lower than about 2,500 Da.

A ligand is a small molecule that can bind to another molecule called a receptor. The ligand can be a steroid, enzyme, protein, aptamer, antibody or other molecule.

A competitive assay is a test where a competitive ligand competes with a target small molecule for binding to a receptor molecule.

A probe is a molecule or group of molecules configured to quantitatively detect a target small molecule in a sample in an assay.

A competitive ligand is a molecule that can be bound to a probe that resembles, or is very similar to, a target ligand; in particular it will bind to a target molecule receptor with a similar affinity as the target molecule.

A signaling molecule or tag is a compound that can be bound to another molecule that either gives off, or can be stimulated to give off, a detectable signal such as a fluorescence (fluorophore), radiation (radio-nucleotide), absorbance (dye) or other detectable indicator.

A linker is a chain-like molecule that can be bound to other molecules on both ends or elsewhere. The linker can be a chemical chain which may be a single or repeating chemical moiety, or it can be a single or double stranded DNA segment, or a combination of both.

A competitive probe is a probe with one or more competitive ligands bound to one or more a signaling molecules, usually through one or more linkers.

### EMBODIMENTS OF THE PRESENT INVENTION

The compounds (I) and (II) of the invention shown above include a ligand (“competitive ligand”) that is linked through a chemical linker to a signaling molecule tag that is detectable (“tag”). Detection in various embodiments can be fluorescent, electrochemiluminescence, radioactive, color or any other technique for detecting the presence and concentration of the tag. Tags may also be linked to, or incorporated within, single and double DNA strands. Incorporation of the tag into a DNA sequence is within the scope of the present invention as well as attaching it to a DNA base or linker. The bifunctional compound can thus competitively bind to a target and simultaneously facilitate quantitative detection of a target molecule. The use of cDNA tags provide modularity that can be tuned for multiplexing and may be released from the DNA sequence to provide additional control to the system.

An example of a probe using double-stranded DNA has been given above and is repeated here for convenience:

(competitive ligand)-LINKER-(DNA)(cDNA)-signaling molecule) (II)

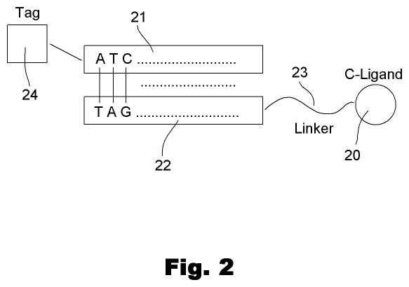

The juxtaposition of DNA and cDNA in the above diagram indicates that base pairs are linked by typical DNA hydrogen bonding (A-T, C-G, where A is adenine, T is thymine, C is cytosine, and G is guanine). This is shown in FIG. 2. It should be noted that ATC attached to TAG in FIG. 2 is for example only. Any DNA sequence that is long enough to stay bound under operating temperatures and conditions may be used. For ease in use, the DNA sequence should be short enough that it can be separated when desired. Strand separation can be accomplished by techniques known in the art. This allows for switching of tags and more complete control.

Prepared DNA (single strands) may attached to different competitive ligands for assays and for several different small molecules, such as various different hormones. cDNA strands can be prepared with tags ready for use. Then to complete a batch of probes for a particular assay, it is only necessary to allow the correctly prepared DNA and cDNA strands to link.

It should be noted that the cDNA sequence does not need to be an exact complement of the DNA where all base pairs bind. While, total binding is preferred (exact complement), partial binding is within the scope of the present invention, as long as the partial binding is strong enough to prevent separation of the two DNA strands at maximum operating temperatures and conditions. Partial linking is useful if it is desired to embed or attach a different molecule to the DNA backbone at one or more locations.

In various embodiments of the present invention, any linker may be used, including, but not limited to, chemical linkers and linkers using two or more separate DNA sequences or a combination of both, as long as the competitive ligand of formulas (I) or (II) can bind to the receptor and facilitate detection.

The competitive ligand, can be a small molecule ligand and/or a peptide ligand, that is capable of binding to the immobilized receptor site for detection. As stated, use of the competitive ligand is such that a target small molecule attenuates detection (signal levels are lower with higher concentrations of the target molecule).

As stated under definitions, the term “small molecule” means that the molecule is typically non-peptidyl, i.e., it is not generally considered a peptide, if it comprises fewer than 4 amino acids, or if it is a steroid, hormone or other molecule with low molecular weight. A small molecule typically has a molecular weight that is lower than about 2,500 Da. Examples of small target molecules of considerable interest are Estrogen, Progesterone and Testosterone. The scope of the present invention is not limited to these hormones. Also larger molecules then what has been defined as a “small molecule” are within the scope of the present invention.

### EMBODIMENTS OF THE PRESENT INVENTION

As previously stated, the basic model for the probe of the present invention has a structure similar to:

(competitive ligand)-LINKER-(tag) (I)

wherein the ligand binds to a target such that it competes with a relevant small target molecule; wherein the tag such as, but not limited to, fluorescent labels, proteins such as, but not limited to, HRP or BSA, or DNA with fluorescent labels allows detection; and wherein the LINKER is selected such that it allows for the compound to bind to a receptor and simultaneously facilitate detection.

Also as stated, a non-limiting embodiment of a compound of the invention

(depicted therein as (competitive ligand)-(tag)) (competitive ligand)-LINKER-(DNA) (cDNA-tag) (II)

wherein the ligand binds to a receptor such that a relevant small molecule competes; wherein the DNA binds a complementary DNA (cDNA); wherein the tag (cDNA-tag) such as, but not limited to, biotin, fluorescent labels, or a protein, such as, but not limited to, HRP or BSA allows detection; and wherein the LINKER is selected such that it allows for the compound to bind to target and simultaneously facilitate detection.

It is not necessary to only use to one tag or one competitive ligand. The probes of the present invention can be linked to multiple tags for more complete detection and/or linked to multiple competitive ligands for use with different receptors or for testing for multiple different molecules.

In certain embodiments, the compound of the invention comprises, and/or has the formula:

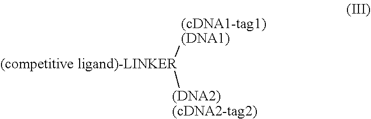

wherein the ligand binds to a target such that a relevant small molecule competes; wherein DNA1 binds cDNA1 and DNA2 binds cDNA2; wherein tag1 such as, but not limited to, biotin, fluorescent labels, or a protein, such as but not limited to HRP or BSA allows detection; wherein tag2 may be the same or different from tag1; wherein the LINKER is selected such that it allows for the compound to bind to target and simultaneously facilitate detection.

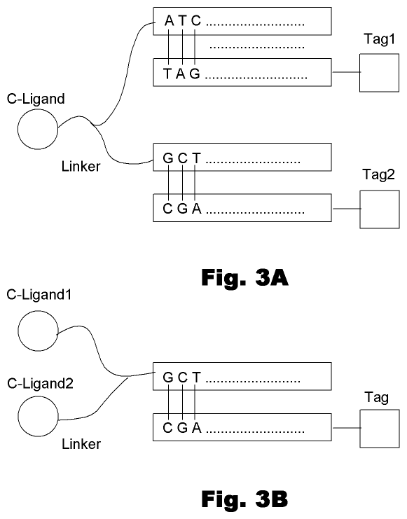

In certain embodiments, the compound of the invention comprises, and/or has the formula:

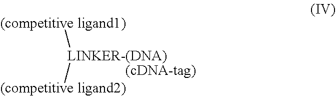

wherein ligand1 binds to target1 such that a relevant small molecule competes; wherein the ligand2 binds to target2 such that a relevant small molecule competes; wherein DNA binds cDNA; wherein the tag such as but not limited to biotin, fluorescent labels, or a protein, such as but not limited to HRP or BSA allows detection; wherein the LINKER is selected such that it allows for the compound to bind to target and simultaneously facilitate detection. FIGS. 3A-3B show embodiments of probes III and IV.

In alternate embodiments both multiple ligands and multiple tags are used.

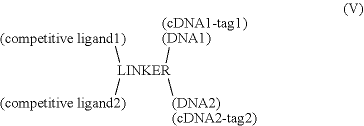

This can be done directly to the linker, or with additional DNA.

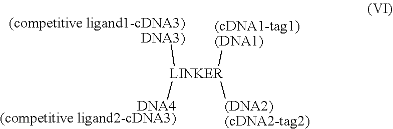

In these embodiments, the LINKER may be a chemical linker, or may itself contain DNA (or both) for example:

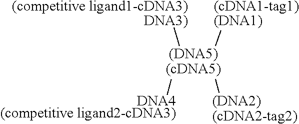

By choosing the sequences DNA1-DNA5 carefully, it is possible to selectively bind and unbind DNA and cDNA parts of these molecules. For example, the different DNA sequences may be chosen to have different melting points. Any technique for selectively binding and unbinding such DNA fragments is within the scope of the present invention.

In various embodiments, the competitive ligand and the tag may both be attached to the 5 ends of the DNA and cDNA. However, it is within the scope of the present invention to reverse this and connect both to the 3′ ends. In either case, the competitive ligand and tag are attached at the two opposite extrema of the DNA-cDNA double strand. As is known in the art, attachment to 5′ end of a single DNA strand is typically made linking to the last phosphate group, while attachment to the 3′ end is typically made by linking to a hydroxy group on the last sugar. Any method of attaching to a DNA strand is within the scope of the present invention.

The DNA strand sequences are typically chosen to be fairly short—in the range of 12-30 mer. The sequences should generally be chosen to avoid hairpins and other undesirable characteristics. Shorter strands generally have less problems in this regard than longer ones. Melting points of the bound strands should be above 40 degrees C., and preferably above 45 degrees C. in order to maintain binding at common laboratory fluid temperatures, for example in Lateral flow and Elisa. However, they should be short enough to allow relatively easy strand separation using known techniques and short enough to prevent undesirable manifestations such as hairpins.

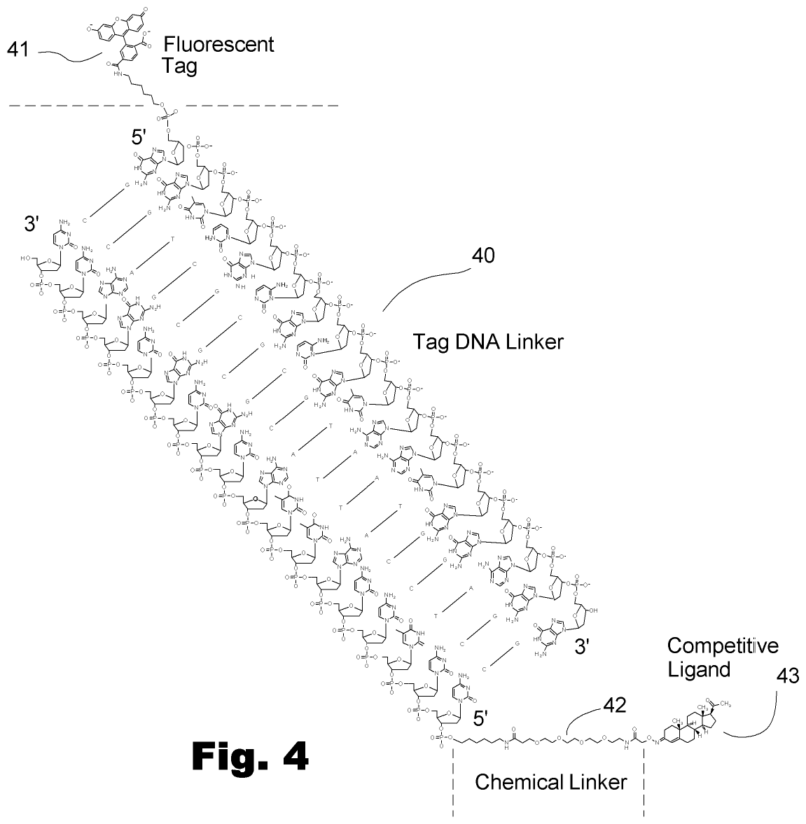

FIG. 4 shows an example type II probe for the hormone progesterone. The linked double stranded DNA center of the probe 40 can be seen. The particular nucleotide sequence shown in the figure is for example only; any sequence is within the scope of the present invention. Reading 3′ to 5, the example sequence is CCA GCG CGC ATT ACC TCC (sequence ID Number= “1”). The other strand is the complement of this sequence. A chemical linker 42 is attached to the 5′ end of the DNA strand (bottom strand in the figure). The length and structure of the linker can be chosen to separate the competitive ligand 43 from the rest of the probe so that the other parts of the probe do not interfere with binding between the competitive ligand and the receptor. The linker 42 is attached to an example competitive ligand for progesterone 43. At the opposite end of the probe, a fluorescent tag 41 is attached to the complementary strand at the 5′ end (top strand in figure). While a particular fluorophore is shown, any type of signaling tag may be used, including, but not limited to, radioisotopes, epitopes, biotin and other fluorophores. The formula for the probe molecule of FIG. 4 is C₂₁₁H₂₆₈N₆₆O₁₁₇P₁₈, and the molecular weight is 6158.30 (neglecting the cDNA-tag).

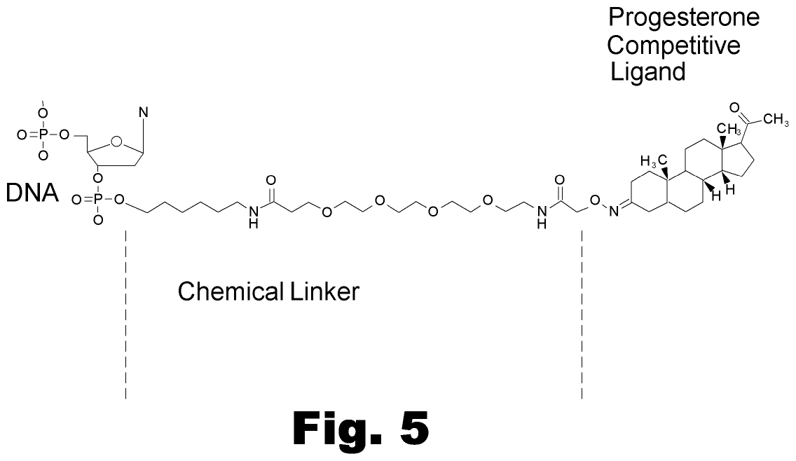

FIG. 5 is a detail of FIG. 4 showing only the example chemical linker and the competitive ligand from FIG. 4. The linker is a short linear section of a repeating moiety chosen to have particular physical and 3-dimensional properties such as stiffness and resistance to kinking or cross-linking. The linker typically separates the competitive ligand from the DNA backbone so that the ligand's properties to the receptor molecule is minimally inhibited by the rest of the probe. The length of the linker, while somewhat variable, should be chosen to provide adequate separation without introducing other undesirable properties. The linker should easily couple to both the DNA strand at one end, and the competitive linker molecule at the other end. The binding of the linker at each end should be stable at normal test temperatures and the test chemical environment.

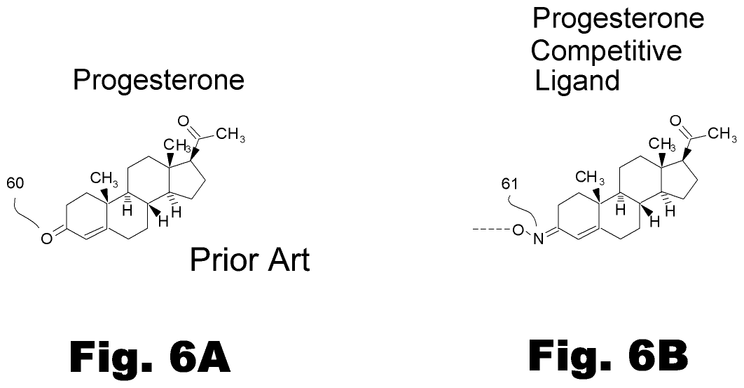

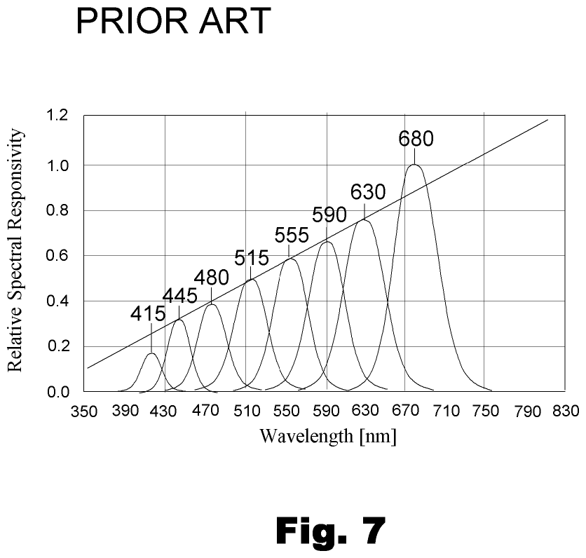

FIG. 6A shows the chemical structure of progesterone, while FIG. 6B shows an example competitive ligand for progesterone. It is clear that in this case, the competitive ligand is simply a progesterone molecule with the 3-carbon bound to nitrogen 61 rather than oxygen 60. Because nitrogen has the ability to bind with a valance of 3 (or 4), where the oxygen only binds with a valance of 2, the nitrogen can link the competitive ligand to the rest of the probe and maintain the receptor binding site recognition to that of the progesterone molecule, particularly with minimal effect on the binding properties of the competitive ligand to the assay receptor molecule. In general, the competitive ligand should maintain the key aspects of the target molecule that permit a strong degree of competitions at the primary binding site.

It is well-known in the field of scientific measurements, that some target signals or responses are very weak, and others are very strong. The entire range of intensities is called dynamic range.

The present invention has one or more signaling parts that signal the presence of a probe molecule on a plate or otherwise. A typical signaling part or molecule can be a fluorophore that, upon stimulation by a particular wavelength of light, emits a second or response light signal of a different wavelength. Prior art table 1 shows the wavelength responses of some common fluorescent signaling molecules.

TABLE 1

| | Catalog/non | | | | | |
|---|---|---|---|---|---|---|
| Fluorophore | catalog | Excitation | | Emission | | Channel |
| 6′-FAM Azid | Catalog | 496 | nm | 516 | nm | |
| Fluorescence dT | Catalog | 495 | nm | 520 | nm | |
| Cy3 | Catalog | 550 | nm | 564 | nm | |
| Cy5 | Catalog | 648 | nm | 668 | nm | |
| TAMRA Azid | Catalog | 546 | nm | 579 | nm | |
| TAMRA | Catalog | 559 | nm | 583 | nm | |
| (NHS Ester) | | | | | | |
| ATT0425 | Non-Catalog | 437 | nm | 483 | nm | |
| Alexa Flour 350 | Non-Catalog | 346 | nm | 442 | nm | |
| Europium Cryptate | Non-Catalog | 320-340 | nm | 615 | nm | F6-F7 |

The fluorophores in the table are excited by light of an excitation wavelength, and emit light at an emission wavelength. Usually, the emission wavelength is longer than the excitation wavelength as can be seen in the table. All of the fluorophores in the table are commercially available prior art products.

An alternate embodiment of the present invention is an anti-sense probe that increases the range of concentration detection of a target antigen. Different fluorophores have different sensitivity; some have low sensitivity, and some have high sensitivity. With a low sensitivity fluorophore a higher concentration of antigen that is bind to the molecule that is carrying the fluorophore is required. This results in reporting strong signals without saturating.

With a high sensitivity fluorophore, very small concentrations of the bound antigen can be detected. With a very low concentration of the antigen, the low sensitivity fluorophore cannot produce enough signal that can be read, but the high sensitivity fluorophore gives a high signal readable signal. Usually, bright fluorophores should be conjugated to antibodies against low expressing cellular targets, whereas fluorophores of low brightness should be used for highly expressed proteins to avoid spillover and loss of resolution and sensitivity.

This embodiment of the present invention uses two (or three or more) different fluorophores on the same anti-sense probe, one with low sensitivity, and one with high sensitivity. The detector can read both simultaneously, and in that manner be able to detect both low and high concentration ranges. The differences between the sensitivity of different fluorophores can be more than 1,000-fold. This allows dual reading of the same sample, with the exactly the same assay, using the ability of the DNA part of the probe to be conjugate to different fluorophores resulting in a very wide dynamic range in a single test.

For example, a probe with two different signaling molecules (Tags) can be similar to:

(competitive ligand)-LINKER-(DNA) (cDNA-)-Tag2

A particular example could be:

The first row is DNA, and the second row is cDNA (the conjugate to the first row).
1) Two fluorophores conjugations:

5′-CCT CCA TTA CGC GCG ACC-3′
(sequence ID Number = “1”).
5′-GGT CGC GCG TAA TGG AGG-3′

C conjugated to Europium Cryptate (680 in FIG. 7)
G conjugated to Alexa Flour 350 (445 in FIG. 7)
2) Three fluorophores conjugations:

5′-CCT CCA TTA CGC GCG ACC-3′
(sequence ID = “1”).
5′-GGT CGC GCC TAA TGG AGG-3′

C conjugate to Europium Cryptate (630 in FIG. 7)
T conjugated to Fluorescence-DT-6′-FAM Azid (515 in FIG. 7)
G conjugated to Alexa Flour 350 (445 in FIG. 7)

It should be noted that the DNA and cDNA sequences shown above are exemplary. Different sequences may be also used, as has been previously described. Typically, the conjugated bases (bases conjugated to a signaling molecule or tag) are conjugated to different species of bases; however, this is not required. Fluorophores conjugated to any base species (A, T, G or C) is within the scope of the present invention.

In a variation of this embodiment, it is also possible to use more than one fluorophore molecule with the same sensitivity on the same probe. They can be identical fluorophore molecules or different fluorophore molecules of similar sensitivity. Reading all the fluorophores in the same assay and calculating the average increases the accuracy of the results.

Generally, with this embodiment, the similar fluorophores can either be separated on the cDNA strand or be next to each other. The use of similar, but not identical, fluorophores is subject to several constraints:
1) a strong overlap between the donor emission spectrum and the acceptor absorption spectrum;
2) if the fluorophore molecules are small, they should be less than 10 nm apart;
3) if the fluorophore molecules are large, they should be greater than 10 nm apart;
4) so that the emission of one fluorophore does not itself trigger a response from a different one requires: a low quantum yield of the potential donor and a small extinction coefficient of a potential acceptor.

FIG. 7 shows a prior art graph of the sensitivities of a group of prior art fluorophores. It can be seen that fluorophore 415 has the lowest sensitivity, while 680 has the highest sensitivity. The emission peaks follow a linear progression of spectral responsivity with the exception of peaks 415 and 680 in FIG. 7.

It is therefore expected that both the 445,630 probe or the 445, 515, 630 probe given in the above examples have a very wide dynamic range.

The probes of the present invention allow fast quantitative measurement of the level of target molecule in an unknown sample. In order to attain accurate quantitative results, a particular probe can be calibrated using known amounts of target molecules in a series of calibration runs. Once a probe type (competitive ligand, linker, DNA and tags) has been calibrated for a particular assay, it should only need minimal recalibration unless there is a major change in the assay process, or the sample preparation.

It should be understood that in addition to DNA and complimentary cDNA strands, RNA strands may also be used where Uracil (U) is substituted for Thymine (T) and binds with Adenine (A) in the complimentary strand.

Unless defined otherwise, all technical and scientific terms used herein have the same meaning as commonly understood by one of ordinary skill in the art to which this invention belongs. Although any methods and materials similar or equivalent to those describe herein can be used in the practice or testing of the present invention, specific methods and materials are described.

The probes of the present invention allow fast quantitative measurement of the level of target molecule in an unknown sample. In order to attain accurate quantitative results, a particular probe can be calibrated using known amounts of target molecules in a series of calibration runs. Once a probe type (competitive ligand, linker, DNA and tags) has been calibrated for a particular assay, it should only need minimal recalibration unless there is a major change in the assay process, or the sample preparation.

Unless defined otherwise, all technical and scientific terms used herein have the same meaning as commonly understood by one of ordinary skill in the art to which this invention belongs. Although any methods and materials similar or equivalent to those describe herein can be used in the practice or testing of the present invention, specific methods and materials are described.

### Example Probe DNA Sequences

NOTE:
This is not a “sequence listing or
“Sequence Listing XML file. The
Sequence Listing is in the XML file
titled” 68272US06.xml”
5′-CCTCCATTA CGCGCGACC-3′
(sequence ID = “1”).
Length: 18 mer
Hairpin: NO
Self dimer: 4 BASES
Melting point: 58.9° C.
5′-GGTATTATG CGCGAAGGAA-3′
(sequence ID Number = “2”).
Length: 19 mer
Hairpin:NO
Self dimer: 4 bases
Melting point: 52.6° C.
5′-CTA TTA GC GCC GTC CTC C-3′
(sequence ID Number = “3”).
Length: 18 mer
Hairpin: NO
Self dimer: not at room temp
Melting point: 55.1° C.
5′-CTT CTC GCG TTA TTC-3′
(sequence ID = “4”).
Length: 15 mer
Hairpin: NO
Self dimer: not at room temp
Melting point: 43.4° C.
5′-GTA TTA TGC GCG GAG-3′
(sequence ID = “5”).
Length: 15 mer
Hairpin: NO
Self dimer: 3 BASES
Melting point: 44.2° C.
5′-TAT CGC GAC ATA AC-3′
(sequence ID Number = “6’).
Length: 14 mer
Hairpin: NO
Self dimer: 6 BASES
Melting point: 40.4° C.
(sequence ID Number = “7”)
5′-CCT TTC GCG TAT CC-3′.
Length: 14 mer
Hairpin: NO
Self dimer: 4 BASES
Melting point: 45.8° C.
5′-TTC GCG ATC ATC CAC CTT CCT T-3′
(sequence ID Number = “8”).
Length: 22 mer
Hairpin: NO
Self dimer: 6 BASES
Melting point: 58.6° C.
5′-TAACGCGACAAAAC-3′
(sequence ID Number = “9”).
Length: 14 mer
Hairpin: NO
Self dimer: 4 BASES
Melting point: 42.5° C.
5′-CCT TTC GCG TAT CCT TCC-3′
(sequence ID Number = “10”).
Length: 18 mer
Hairpin: NO
Self dimer: 4 BASES
Melting point: 53° C.
5′-CCT TTC GCG TAT CCT T-3′
(sequence ID Number = “11”).
Length: 16 mer
Hairpin: NO
Self dimer: 4 BASES
Melting point: 48.7° C.
5′-CTA TTA TGC GCC GTC CTC C-3′
(sequence ID Number: “12”).
Length: 19 mer
Hairpin: NO
Self dimer: not at room temp
Melting point: 55.4° C.
5′-TAT CGC GAC ATA ACC AA-3′
(sequence ID Number = “13”).
Length: 17 mer
Hairpin: Not on RT
Self dimer: 6 BASES
Melting point: 47.7° C.
5′-TAT CGC GAC ATA ACA A-3′
(sequence ID Number = “14”).
Length: 16 mer
Hairpin: Not on RT
Self dimer: 6 BASES
Melting point: 44.3° C.

Several examples of DNA linker sequences have been listed. The scope of the present invention is not limited to these examples.

As stated above, the applicant is providing a “Sequence Listing XML” entitled “68272US06.xml” as part of this disclosure and as incorporated by reference on page 1 of this disclosure.

Several descriptions and illustrations have been presented to aid in understanding the present invention. One with skill in the art will realize that numerous changes and variations may be made without departing from the spirit of the invention. Each of these changes and variations is within the scope of the present invention.

1. A biological probe configured to competitively assay small molecules, the probe comprising:
a first probe part comprising a single strand DNA sequence, the first probe part attached to at least one competitive ligand competitive to a target small molecule;
a second probe part comprising the single strand cDNA complement of the single strand DNA sequence of the first probe part, the second probe part comprising first and second fluorescent signaling molecules each conjugated to a particular base in the single strand cDNA complement;
wherein the first signaling molecule has a higher sensitivity than the second signaling molecule;
wherein the first signaling molecule is conjugated proximate 5′-end of the cDNA complement, and the second signaling molecule is conjugated proximate the 3′-end of the cDNA complement; and
wherein the first signaling molecule is Europium Cryptate, and the second signaling molecule is Alexa Flour 350.

2. The biological probe of claim 1, wherein the first and second signaling molecules are conjugated to different nucleic acid base species.

3. The biological probe of claim 1, wherein the first signaling molecule is conjugated to C, and the second signaling molecule is conjugated to G.

4. The biological probe of claim 1, wherein the single strand DNA sequence of the first probe part is selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, SEQ. ID. NO. 9, SEQ. ID. NO. 10, SEQ. ID. NO. 11, SEQ. ID. NO. 12, SEQ. ID. NO. 13, and SEQ. ID. NO. 14.

5. The biological probe of claim 1, wherein the target small molecule is a hormone.

6. The biological probe of claim 1, wherein the target small molecule is progesterone.

7. A biological probe configured to competitively assay small molecules, the probe comprising:
a first probe part comprising a single strand DNA sequence, the first probe part attached to at least one competitive ligand competitive to a target small molecule;
a second probe part comprising the single strand cDNA complement of the single strand DNA sequence of the first probe part, the second probe part comprising first, second, and third fluorescent signaling molecules each conjugated to a particular base in the single strand cDNA complement;
wherein the first signaling molecule has a higher sensitivity than the second signaling molecule, and the second signaling molecule has a higher sensitivity than the third signaling molecule;
wherein the first signaling molecule is conjugated proximate 5′-end of the cDNA complement, the third signaling molecule is conjugated proximate 3′-end of the cDNA complement, and the second signaling molecule is conjugated to the cDNA complement between the first and third signaling molecules; and
wherein the first signaling molecule is Europium Cryptate, the second signaling molecule is Fluorescence DT, and the third signaling molecule is Alexa Flour 350.

8. The biological probe of claim 7, wherein the first, second, and third signaling molecules are conjugated to different nucleic acid base species.

9. The biological probe of claim 7, wherein the first signaling molecule is conjugated to C, the second signaling molecule is conjugated to T, and the third signaling molecule is conjugated to G.

10. The biological probe of claim 7, wherein the single strand DNA sequence of the first probe part is selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, SEQ. ID. NO. 9, SEQ. ID. NO. 10, SEQ. ID. NO. 11, SEQ. ID. NO. 12, SEQ. ID. NO. 13, and SEQ. ID. NO. 14.

11. The biological probe of claim 7, wherein the target small molecule is a hormone.

12. The biological probe of claim 7, wherein the target small molecule is progesterone.

13. A biological probe configured to competitively assay small molecules, the probe comprising:
a first probe part comprising a single strand DNA sequence, the first probe part attached to at least one competitive ligand competitive to a target small molecule;
a second probe part comprising the single strand cDNA complement of the single strand DNA sequence of the first probe part, the second probe part comprising first and second fluorescent signaling molecules each conjugated to a particular base in the single strand cDNA complement;
wherein the first and second signaling molecules have overlapping donor emission spectrums and overlapping acceptor absorption spectrums; and
wherein the first signaling molecule is conjugated proximate 5′-end of the cDNA complement, and the second signaling molecule is conjugated proximate the 3′-end of the cDNA complement.

14. The biological probe of claim 13, wherein the first and second signaling molecules are conjugated to different nucleic acid base species.

15. The biological probe of claim 13, wherein the single strand DNA sequence of the first probe part is selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, SEQ. ID. NO. 9, SEQ. ID. NO. 10, SEQ. ID. NO. 11, SEQ. ID. NO. 12, SEQ. ID. NO. 13, and SEQ. ID. NO. 14.

16. The biological probe of claim 13, wherein the target small molecule is a hormone.

17. The biological probe of claim 13, wherein the target small molecule is progesterone.


SEQUENCE LISTING (14 sequences)

SEQ ID NO: 1 - DNA, 18 bp, synthetic construct
  misc_feature 1..18: note = Sythentic Probe
  source 1..18: mol_type = other DNA
cctccattacgcgcgacc

SEQ ID NO: 2 - DNA, 19 bp, synthetic construct
  misc_feature 1..19: note = Synthetic Probe
  source 1..19: mol_type = other DNA
ggtattatgcgcgaaggaa

SEQ ID NO: 3 - DNA, 18 bp, synthetic construct
  misc_feature 1..18: note = Synthetic Probe
  source 1..18: mol_type = other DNA
ctattagcgccgtcctcc

SEQ ID NO: 4 - DNA, 15 bp, synthetic construct
  misc_feature 1..15: note = Synthetic Probe
  source 1..15: mol_type = other DNA
cttctcgcgttattc

SEQ ID NO: 5 - DNA, 15 bp, synthetic construct
  misc_feature 1..15: note = Synthetic Probe
  source 1..15: mol_type = other DNA
gtattatgcgcggag

SEQ ID NO: 6 - DNA, 14 bp, synthetic construct
  misc_feature 1..14: note = Synthetic Probe
  source 1..14: mol_type = other DNA
tatcgcgacataac

SEQ ID NO: 7 - DNA, 14 bp, synthetic construct
  misc_feature 1..14: note = Artificial Probe
  source 1..14: mol_type = other DNA
cctttcgcgtatcc

SEQ ID NO: 8 - DNA, 22 bp, synthetic construct
  misc_feature 1..22: note = Synthetic Probe
  source 1..22: mol_type = other DNA
ttcgcgatcatccaccttcctt

SEQ ID NO: 9 - DNA, 14 bp, synthetic construct
  misc_feature 1..14: note = Artificial Probe
  source 1..14: mol_type = other DNA
taacgcgacaaaac

SEQ ID NO: 10 - DNA, 18 bp, synthetic construct
  misc_feature 1..18: note = Synthetic Probe
  source 1..18: mol_type = other DNA
cctttcgcgtatccttcc

SEQ ID NO: 11 - DNA, 16 bp, synthetic construct
  misc_feature 1..16: note = Synthetic Probe
  source 1..16: mol_type = other DNA
cctttcgcgtatcctt

SEQ ID NO: 12 - DNA, 19 bp, synthetic construct
  misc_feature 1..19: note = Synthetic Probe
  source 1..19: mol_type = other DNA
ctattatgcgccgtcctcc

SEQ ID NO: 13 - DNA, 17 bp, synthetic construct
  misc_feature 1..17: note = Synthetic Probe
  source 1..17: mol_type = other DNA
tatcgcgacataaccaa

SEQ ID NO: 14 - DNA, 16 bp, synthetic construct
  source 1..16: mol_type = other DNA
  misc_feature 1..16: note = Synthetic Probe
tatcgcgacataacaa